(19) United States
(12) Patent Application Publication
(10) Pub. No.: US 2008/0014257 A1
(43) Pub. Date: Jan. 17, 2008

(54) Oral dosage forms

(75) Inventors: Ping He, Miramar, FL (US); Damodar Patel, Elmwood Park, NJ (US); Suketu Sanghvi, Kendall Park, NJ (US); Robert Femia, Kinnelon, NJ (US)

Correspondence Address: FOLEY AND LARDNER LLP;SUITE 500, 3000 K STREET NW, WASHINGTON, DC, 20007, US

(73) Assignee: Par Pharmaceutical, Inc.

(21) Appl. No.: 11/486,375

(22) Filed: Jul. 14, 2006

(51) Int. Cl. A61K 9/52; A61K 31/65; A61K 31/4439

(52) U.S. Cl. 424/457; 514/338; 514/152

(57) ABSTRACT

This invention relates to an oral dosage form of a pharmaceutically active ingredient comprising: (a) an outer capsule and (b) non-uniform pellets, having a non-uniform shape and/or size, contained within the capsule, wherein the pellets comprise a compressed powder comprising a pharmaceutically active ingredient. In one embodiment the active ingredient is selected from the group consisting of doxycycline, omeprazole, esomeprazole, and propafenone. Pharmaceutical formulations of the active ingredients as well as methods and tools for making the oral dosage form are also described.

### FIELD OF INVENTION

The present invention relates generally to the field of pharmaceutical compositions for oral administration, including controlled release compositions.

### BACKGROUND OF THE INVENTION

An area of current research focus in the pharmaceutical industry is the development of methods for the controlled or sustained release of drugs. Such methods obviate certain problems associated with traditional methods for administering drugs, such as non-compliance of patients with a prescribed medication schedule, the need for frequent administrations, and fluctuating concentrations of the drug in the body. Methods for sustained or controlled drug release typically utilize an implanted device, such as an osmotic pump, or a drug dispersed in a biocompatible polymer matrix, which can be implanted, administered orally, or injected.

Attempts to develop sustained-release formulations have included the use of a variety of biodegradable and non-biodegradable polymer (e.g., poly(lactide-co-glycolide)) microparticles containing the active ingredient, and a variety of techniques are known by which active agents can be incorporated into polymeric microspheres. In these formulations, the release profile for the active ingredient may be continuous or discontinuous, and in some cases the initial level of active ingredient release is higher or lower than desired for optimal efficacy.

Therefore, the need still exists for pharmaceutical compositions for oral administration that yield a desired drug release profile, including a controlled release or extended release profile.

Propafenone hydrochloride is an anti-arrythmic agent sold in the United States and elsewhere under the trade name Rythmol®, in the form of immediate-release tablets. The usual dosing schedule is three times daily. In early 2004, propafenone hydrochloride also became available in the United States and elsewhere under the trade name Rythmol SR® in the form of sustained release capsules. Because the release from Rythmol SR® is more gradual, the dosing schedule for Rythmol SR® is only twice daily. Rythmol SR® capsules are made in accordance with the disclosure of U.S. Pat. No. 5,681,588. As explained in that patent, the Rythmol SR® formulation comprises gelatin capsules filled with microtablets comprising propafenone hydrochloride with little or no excipients added. The described microtablets have a height and diameter which are each 1-3 mm, with the active ingredient constituting from 81 to 99.9% of the weight of the microtablet.

The microtablets contained in the Rythmol SR® capsules have a uniform diameter of 2 mm, a propafenone hydrochloride content of 6.25 mg, and a total weight of 6.5 mg per microtablet. Rythmol SR® capsules are produced on conventional rotary tablet presses. Rotary tablet presses produce a number of tablets per minute that is equal to the number of rotations of the press per minute multiplied by the number of tooling stations. Hence, for production of microtablets needed to fill a given number of capsules, the production time on a tablet press is directly proportional to the number of microtablets needed. Furthermore, the tooling needed to produce microtablets of 2 mm diameter is relatively fragile and easily broken. Tabletting rates can be increased by using multi-tip tooling, but such tooling is relatively expensive and also relatively fragile.

There exists a need for improved controlled release or sustained release dosage forms of a pharmaceutically active ingredient, including but not limited to doxycycline, omeprazole, esomeprazole, and propafenone. The present invention satisfies this need.

### SUMMARY OF THE INVENTION

The present invention provides pharmaceutical compositions for oral administration.

In accordance with one embodiment, the invention provides an oral dosage form of a pharmaceutically active ingredient comprising: (a) an outer capsule and (b) non-uniform pellets, having a non-uniform shape and/or size, comprised within the capsule, wherein the pellets comprise a compressed powder comprising a pharmaceutically active ingredient. In specific embodiments, the active ingredient is selected from the group consisting of doxycycline, omeprazole, esomeprazole, and propafenone.

The non-uniform pellets may have a diameter of from about 1 mm to about 3 mm or have a maximum and a minimum diameter, each of which is independently from about 1 mm to about 3 mm. In addition, the non-uniform pellets can be primarily spherical, cubic, cylindrical, or irregular.

In some embodiments the non-uniform pellets are coated with a pharmaceutically acceptable coating material. In one embodiment, the coating material comprises Opadry colors, or a material selected from a pH-dependent polymer and a pH-independent polymer. In one embodiment, the pH-dependent polymer is selected from the group consisting of methacrylic acid/methyl methacrylate copolymers (Eudragit L 100, Eudragit S 100), methacrylic acid/ethyl acrylate copolymers (Eudragit L 30D 55), methacrylic acid/methyl acrylate and methyl methacrylate copolymers (Eudragit FS 30D), cellulose acetate phthalate, cellulose acetate trimellitate, hydroxypropyl methylcellulose acetate succinate, hydroxypropyl methylcellulose phthalate, povinyl acetate phthalate and shellac. In another embodiment, the pH-independent polymer is selected from the group consisting of ethylcellulose, methacrylic ester copolymers (Eudragit NE 30D), and ammonio methacrylate copolymers (Eudragit RL 30D and Eudragit RS 30D).

In one specific embodiment the compressed powder comprises: (a) about 78.5 to about 79.5% by weight propafenone; (b) about 4.5 to about 5.5% by weight povidone; (c) about 4.5 to about 5.5% by weight ethyl cellulose; (d) about 9.5 to about 10.5% by weight lactose anhydrous, and (e) about 0.5 to about 1.5% by weight magnesium stearate. In another specific embodiment, the powder comprises: (a) about 78.5 to about 79.5% by weight propafenone; (b) about 4.5 to about 5.5% by weight povidone; (c) about 2.5 to about 3.5% by weight glyceryl behenate; (d) about 11.5 to about 12.5% by weight lactose anhydrous, and (e) about 0.5 to about 1.5% by weight magnesium stearate.

In another embodiment, the pellets are made by a process comprising: (a) compressing a powder comprising that pharmaceutically active ingredient into compressed slugs and (b) breaking the slugs into non-uniform pellets. The compressing step may comprise roller compaction. Optionally, the process may further comprise subsequent to step (a) and prior to step (b), scoring at least one surface of the slugs. In one particular embodiment, the scoring is effected with the compressing step. In one specific embodiment, the scoring is up to a depth of about 95% of the thickness of the slug.

The invention also provides a method of producing an oral dosage form of a pharmaceutically active ingredient, comprising: (a) compressing a powder comprising a pharmaceutically active ingredient into slugs, (b) breaking the slugs into non-uniform pellets, and (c) encapsulating the non-uniform pellets in a capsule. In one embodiment, the compressing step comprises roller compaction. In addition, the method may further comprise, subsequent to step (a) and prior to step (b), scoring at least one surface of the slugs. In one particular embodiment, the scoring is effected with the compressing step. In one specific embodiment, the scoring comprises scoring up to about 95% of the thickness of the slug. In addition, subsequent to step (b) and prior to step (c), the method may comprise coating the non-uniform pellets with a pharmaceutically acceptable coating material.

The invention also provides an apparatus for making compressed powder slugs that are scored for breaking into pellets, comprising tooling that is adapted to compress powder into a slug, wherein at least one surface of the tooling is adapted to form scoring on at least one surface of the slug, for breaking the slug into pellets. In one embodiment, the apparatus comprises a die, a lower punch, and an upper punch. In one specific embodiment, at least one of the upper punch and the lower punch is adapted to form scoring on a surface of the slug. In one embodiment, both the upper punch and the lower punch are adapted to form scoring on upper and lower surfaces of the slug. In one particular embodiment, the upper punch and the lower punch are adapted to form scoring of different depths on upper and lower surfaces of the slug, respectively.

In one embodiment of the apparatus, the scoring is adapted for breaking the slug into at least two pellets. In another embodiment of the apparatus, the scoring is adapted for breaking the slug into at least twenty pellets. In one embodiment of the apparatus, the tooling is adapted to form a single slug. In another embodiment of the apparatus, the tooling is adapted to form multiple slugs.

The invention also provides a punch for making compressed powder slugs that are scored for breaking into pellets, comprising a device adapted to form scoring on a surface of the slug for breaking the slug into pellets.

Both the foregoing general description and the following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 illustrates the dissolution profile for a doxycycline oral dosage form.

FIG. 2 illustrates the dissolution profile for an esomeprazole oral dosage form.

FIG. 3 illustrates the dissolution profile of a propafenone oral dosage form.

FIG. 4 illustrates the dissolution profiles of two propafenone oral dosage forms according to one embodiment of the invention (and a reference sample) at pH 4.5.

FIG. 5 illustrates the dissolution profile of two propafenone oral dosage forms according to one embodiment of the invention (and a reference sample) at pH 6.8.

FIG. 6 is a sectional view showing the forces that develop during powder compaction.

FIG. 7a is a sectional view showing loading of powder into a tooling set.

FIG. 7b is a sectional view showing a first stage of powder compaction into a slug.

FIG. 7c is a sectional view showing complete powder compaction into a slug.

FIG. 7d is a sectional view showing ejection of a slug from the tooling set.

FIG. 8 is a sectional view of a tooling part and an exemplary slug.

FIG. 9a illustrates a first tooling design and a slug produced by the first tooling design.

FIG. 9b illustrates a second tooling design and a slug produced by the second tooling design.

FIG. 9c illustrates a third tooling design and a slug produced by the third tooling design.

FIG. 10 illustrates a slug formed with a tooling design, according to one embodiment of the invention.

FIG. 11 illustrates pellets formed from a slug, according to one embodiment of the invention.

FIG. 12 illustrates pellets formed from a slug, according to on embodiment of the invention.

### DETAILED DESCRIPTION

The present invention provides novel pharmaceutical compositions for oral administration, such as oral dosage forms comprising capsules comprising non-uniform pellets, having a non-uniform shape and/or size, and methods of making the same. In some embodiments, the capsules provide controlled delivery of the pharmaceutically active ingredient.

One aspect of the present invention relates to the fact that different sized and shaped pellets have different release rates for the active ingredient, depending, for example, on the surface area of the pellet. Thus, dosage forms of the present invention that comprise a capsule comprising non-uniform pellets can provide controlled and/or extended release of the active ingredient.

A. Definitions

Unless defined otherwise, the terms used herein are intended to have their ordinary meaning in the art. The present invention is described herein using several definitions, as set forth below and throughout the application.

“About” will be understood by persons of ordinary skill in the art and will vary to some extent on the context in which the term is used. If there are uses of the term which are not clear to persons of ordinary skill in the art given the context in which it is used, “about” will mean up to plus or minus 10% of the particular term.

Unless otherwise specified, “a,” “a” or “the” designates one or more, and words used in the singular also include the plural.

As used herein, “compressed tablets”, “micro-tabs”, “micro-tablets”, and “tablets” refer to a compressed powder comprising an active ingredient where the shape of the tablet is regular, such as round, cubic, cylindrical, spherical, or any other regular shape. These tablets may be comprised within a capsule, where each tablet within a capsule has a substantially identical size and shape. In most cases, the size of the compressed tablets ranges from about 1 mm to about 6 mm. Tablets are typically compressed with a rotary tablet press or other similar machinery known to one of skill in the art.

As used herein, “compressed pellets” and “pellets” refer to a compressed powder comprising an active ingredient where the shape of the pellet may be regular or irregular. Of particular relevance to the invention are non-uniform pellets. Like tablets, pellets may be comprised within capsules for oral administration. In accordance with one aspect of the invention, a given capsule will comprise pellets of different shapes and sizes. As discussed in more detail below, pellets useful in the present invention can be made from slugs or tablets, where the slugs or tablets are broken into smaller units (e.g., pellets) in any pharmaceutically acceptable manner known to those skilled in the art.

As used herein, “slugs” refer to a compressed powder with any shape.

As used herein, “extended release” refers to an oral dosage form that effects delivery of an active ingredient over an extended period of time. For example, a dosage form of the present invention may release active ingredient over a period of time of at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours, at least about 16 hours, at least about 17 hours, at least about 18 hours, at least about 19 hours, at least about 20 hours, at least about 21 hours, at least about 22 hours, at least about 23 hours, or at least about 24 hours. In accordance with one embodiment, a dosage form of the present invention releases active ingredient over a period of time of about 12 hours, such that the dosage form need only be administered about every 12 hours for continuous therapeutic effect.

As used herein, “controlled release” refers to an oral dosage form that effects delivery of an active ingredient under certain conditions. For example, a dosage form of the present invention may release active agent under certain pH, salt, or other chemical conditions.

As used herein, “non-uniform pellets” refers to pellets having a non-uniform shape and/or size, such that a sample of pellets comprises individual pellets that exhibit variation in shape and/or size compared to other pellets in the sample.

B. Pharmaceutically Active Ingredients

In the embodiments of the present invention that include pharmaceutically active ingredients, any pharmaceutically active ingredients can be used. This is because the present invention is not limited to a particularly active ingredient or class of active ingredients, but is useful with regard to a wide variety of active ingredients.

In one embodiment, the active ingredient agent is hydrophobic. Hydrophobic active ingredients are compounds with little or no water solubility. Intrinsic water solubilities (i.e., water solubility of the unionized form) for hydrophobic active ingredients are less than about 1% by weight, and typically less than about 0.1% or 0.01% by weight. In a particular aspect of this embodiment, the active ingredient is a hydrophobic drug. In other particular aspects, the active ingredient is a nutrient, a cosmeceutical, a diagnostic agent, or a nutritional agent.

Suitable hydrophobic active ingredients are not limited by therapeutic category, and can be, for example, analgesics, anti-inflammatory agents, antihelminthics, anti-arrhythmic agents, anti-bacterial agents, anti-viral agents, anti-coagulants, anti-depressants, anti-diabetics, anti-epileptics, anti-fungal agent, anti-gout agents, anti-hypertensive agents, anti-malarials, anti-migraine agents, anti-muscarinic agents, anti-neoplastic agents, erectile dysfunction improvement agents, immunosuppresants, anti-protozoal agents, anti-thyroid agents, anxiolytic agents, sedatives, hypnotics, neuroleptics, beta-blockers, cardiac inotropic agents, corticosteroids, diuretics, anti-parkinsonian agents, gastrointestinal agents, histamine receptor antagonists, keratolytics, lipid regulating agents, anti-anginal agents, COX-2 inhibitors, leukotriene inhibitors, macrolides, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof.

Specific, non-limiting examples of suitable hydrophobic active ingredients include but are not limited to acetretin, albendazole, albuterol, aminoglutethimide, amiodarone, amlodipine, amphetamine, amphotericin B, atorvastatin, atovaquone, azithromycin, baclofen, beclomethasone, benezepril, benzonatate, betamethasone, bicalutanide, budesonide, bupropion, busulfan, butenafine, calcifediol, calcipotriene, calcitriol, camptothecin, candesartan, capsaicin, carbamezepine, carotenes, celecoxib, cerivastatin, cetirizine, chlorpheniramine, cholecalciferol, cilostazol, cimetidine, cinnarizine, ciprofloxacin, cisapride, clarithromycin, clemastine, clomiphene, clomipramine, clopidogrel, codeine, coenzyme Q10, cyclobenzaprine, cyclosporin, danazol, dantrolene, dexchlorpheniramine, diclofenac, dicoumarol, digoxin, dehydroepiandrosterone, dihydroergotamine, dihydrotachysterol, dirithromycin, donezepil, efavirenz, eprosartan, ergocalciferol, ergotamine, essential fatty acid sources, esomprazole, etodolac, etoposide, famotidine, fenofibrate, fentanyl, fexofenadine, finasteride, fluconazole, flurbiprofen, fluvastatin, fosphenytoin, frovatriptan, furazolidone, gabapentin, gemfibrozil, glibenclamide, glipizide, glyburide, glimepiride, griseofulvin, halofantrine, ibuprofen, irbesartan, irinotecan, isosorbide dinitrate, isotretinoin, itraconazole, ivermectin, ketoconazole, ketorolac, lamotrigine, lansoprazole, leflunomide, lisinopril, loperamide, loratadine, lovastatin, L-thryroxine, lutein, lycopene, medroxyprogesterone, mifepristone, mefloquine, megestrol acetate, methadone, methoxsalen, metronidazole, miconazole, midazolam, miglitol, minoxidil, mitoxantrone, montelukast, nabumetone, nalbuphine, naratriptan, nelfinavir, nifedipine, nisoldipine, nilutanide, nitrofurantoin, nizatidine, omeprazole, oprevelkin, oestradiol, oxaprozin, paclitaxel, pantoprazole, paracalcitol, paroxetine, pentazocine, pioglitazone, pizofetin, pravastatin, prednisolone, probucol, progesterone, pseudoephedrine, pyridostigmine, rabeprazole, raloxifene, repaglinide, rifabutine, rifapentine, rimexolone, ritanovir, rizatriptan, rofecoxib, rosiglitazone, saquinavir, sertraline, sibutramine, sildenafil citrate, simvastatin, sirolimus, spironolactone, sumatriptan, tacrine, tacrolimus, tamoxifen, tamsulosin, targretin, tazarotene, telmisartan, teniposide, terbinafine, terazosin, tetrahydrocannabinol, tiagabine, ticlopidine, tirofibran, tizanidine, topiramate, topotecan, toremifene, tramadol, tretinoin, troglitazone, trovafloxacin, ubidecarenone, valsartan, venlafaxine, verteporfin, vigabatrin, vitamin A, vitamin D, vitamin E, vitamin K, zafirlukast, zileuton, zolmitriptan, zolpidem, and zopiclone. Of course, salts, isomers, and derivatives of the above-listed hydrophobic active ingredients may also be used, as well as mixtures thereof.

In another embodiment, the active ingredient is hydrophilic compound. Amphiphilic compounds are also included within the class of hydrophilic active ingredients. Apparent water solubilities for hydrophilic active ingredients are greater than about 0.1% by weight, and typically greater than about 1% by weight. In a particular aspect of this embodiment, the hydrophilic active ingredient is a hydrophilic drug. In other particular aspects, the hydrophilic active ingredient is a cosmeceutical, a diagnostic agent, or a nutritional agent.

Suitable hydrophilic active ingredients are not limited by therapeutic category, and can be, for example, analgesics, anti-inflammatory agents, antihelminthics, anti-arrhythmic agents, anti-bacterial agents, anti-viral agents, anti-coagulants, anti-depressants, anti-diabetics, anti-epileptics, anti-ftngal agent, anti-gout agents, anti-hypertensive agents, anti-malarials, anti-migraine agents, anti-muscarinic agents, anti-neoplastic agents, erectile dysfunction improvement agents, immunosuppresants, anti-protozoal agents, anti-thyroid agents, anxiolytic agents, sedatives, hypnotics, neuroleptics, beta-blockers, cardiac inotropic agents, corticosteroids, diuretics, anti-parkinsonian agents, gastro-intestinal agents, histamine receptor antagonists, keratolytics, lipid regulating agents, anti-anginal agents, Cox-2 inhibitors, leukotriene inhibitors, macrolides, muscle relaxants, anti-osteoporosis agents, anti-obesity agents, cognition enhancers, anti-urinary incontinence agents, nutritional oils, anti-benign prostate hypertrophy agents, essential fatty acids, non-essential fatty acids, and mixtures thereof

Specific, non-limiting examples of suitable hydrophilic active ingredients include but are not limited to acarbose; acyclovir; acetyl cysteine; acetylcholine chloride; alatrofloxacin; alendronate; alglucerase; amantadine hydrochloride; ambenomium; amifostine; amiloride hydrochloride; aminocaproic acid; amphotericin B; antihemophilic factor (human); antihemophilic factor (porcine); antihemophilic factor (recombinant); aprotinin; asparaginase; atenolol; atracurium besylate; atropine; azithromycin; aztreonam; BCG vaccine; bacitracin; becalermin; belladona; bepridil hydrochloride; bleomycin sulfate; calcitonin human; calcitonin salmon; carboplatin; capecitabine; capreomycin sulfate; cefamandole nafate; cefazolin sodium; cefepime hydrochloride; cefixime; cefonicid sodium; cefoperazone; cefotetan disodium; cefotaxime; cefoxitin sodium; ceftizoxime; ceftriaxone; cefuroxime axetil; cephalexin; cephapirin sodium; cholera vaccine; chorionic gonadotropin; cidofovir; cisplatin; cladribine; clidinium bromide; clindamycin and clindamycin derivatives; ciprofloxacin; clodronate; colistimethate sodium; colistin sulfate; corticotropin; cosyntropin; cromolyn sodium; cytarabine; dalteparin sodium; danaparoid; desferrioxamine; denileukin diftitox; desmopressin; diatrizoate meglumine and diatrizoate sodium; dicyclomine; didanosine; dirithromycin; dopamine hydrochloride; dornase alpha; doxacurium chloride; doxorubicin; etidronate disodium; enalaprilat; enkephalin; enoxaparin; enoxaparin sodium; ephedrine; epinephrine; epoetin alpha; erythromycin; esmolol hydrochloride; factor IX; famciclovir; fludarabine; fluoxetine; foscamet sodium; ganciclovir; granulocyte colony stimulating factor; granulocyte-macrophage stimulating factor; recombinant human growth hormones; bovine growth hormone; gentamycin; glucagon; glycopyrolate; gonadotropin releasing hormone and synthetic analogs thereof; GnRH; gonadorelin; grepafloxacin; haemophilus B conjugate vaccine; Hepatitis A virus vaccine inactivated; Hepatitis B virus vaccine inactivated; heparin sodium; indinavir sulfate; influenza virus vaccine; interleukin-2; interleukin-3; insulin-human; insulin lispro; insulin procine; insulin NPH; insulin aspart; insulin glargine; insulin detemir; interferon alpha; interferon beta; ipratropium bromide; ifosfamide; Japanese encephalitis virus vaccine; lamivudine; leucovorin calcium; leuprolide acetate; levofloxacin; lincomycin and lincomycin derivatives; lobucavir; lomefloxacin; loracarbef; mannitol; measles virus vaccine; meningococcal vaccine; menotropins; mepenzolate bromide; mesalamine; methenamine; methotrexate; methscopolamine; metformin hydrochloride; metoprolol; mezocillin sodium; mivacurium chloride; mumps viral vaccine; nedocromil sodium; neostigmine bromide; neostigmine methyl sulfate; neurontin; norfloxacin; octreotide acetate; ofloxacin; olpadronate; oxytocin; pamidronate disodium; pancuronium bromide; paroxetine; perfloxacin; pentamidine isethionate; pentostatin; pentoxifylline; periciclovir; pentagastrin; phentolamine mesylate; phenylalanine; physostigmine salicylate; plague vaccine; piperacillin sodium; platelet derived growth factor; pneumococcal vaccine polyvalent; poliovirus vaccine (inactivated); poliovirus vaccine live (OPV); polymyxin B sulfate; pralidoxime chloride; pramlintide; pregabalin; propafenone; propantheline bromide; pyridostigmine bromide; rabies vaccine; residronate; ribavarin; rimantadine hydrochloride; rotavirus vaccine; salmeterol xinafoate; sincalide; small pox vaccine; solatol; somatostatin; sparfloxacin; spectinomycin; stavudine; streptokinase; streptozocin; suxamethonium chloride; tacrine hydrochloride; terbutaline sulfate; thiopeta; ticarcillin; tiludronate; timolol; tissue type plasminogen activator; TNFR:Fc; TNK-tPA; trandolapril; trimetrexate gluconate; trospectinomycin; trovafloxacin; tubocurarine chloride; tumor necrosis factor; typhoid vaccine live; urea; urokinase; vancomycin; valacyclovir; valsartan; varicella virus vaccine live; vasopressin and vasopressin derivatives; vecuronium bromide; vinblastine; vincristine; vinorelbine; vitamin B12; warfarin sodium; yellow fever vaccine; zalcitabine; zanamivir; zolendronate; zidovudine; pharmaceutically acceptable salts, isomers and derivatives thereof; and mixtures thereof.

In specific embodiments of the invention the active ingredient is selected from the group consisting of omeprazole, esomeprazole, propafenone, doxycycline, and pharmaceutically acceptable salts thereof.

C. Oral Dosage Forms

As noted above, one aspect of the invention provides an oral dosage form of a pharmaceutically active ingredient, comprising: (a) an outer capsule and (b) non-uniform pellets comprised within the capsule, wherein the pellets comprise a compressed powder comprising a pharmaceutically active ingredient. Another aspect of the invention provides a method of producing an oral dosage form of a pharmaceutically active ingredient, comprising: (a) compressing a powder comprising a pharmaceutically active ingredient into slugs, (b) breaking the slugs into non-uniform pellets, and (c) encapsulating the non-uniform pellets in a capsule.

1. Compressed Powder

The compressed powder can be made by any means known in the art. For example, the active ingredient can be granulated and dried, and then milled and/or blended into a powder, using methods and equipment that are well-known in the art. The powder can be compressed by any means known in the art, including by the use of tablet presses or roller compactors. Batch processes can be used for large scale production.

The compressed powder may include an amount of active ingredient that will result in an oral dosage form comprising a pharmaceutically effective dose of the ingredient. Those skilled in the art will recognize that this amount may vary with the specific active ingredient, target patient, condition being treated, etc. In addition, the compressed powder may include other pharmaceutically acceptable ingredients, such as those discussed in more detail below.

In one embodiment of the invention, the compressed powder comprises: (a) about 78.5 to about 79.5% by weight propafenone; (b) about 4.5 to about 5.5% by weight povidone; (c) about 4.5 to about 5.5% by weight ethyl cellulose; (d) about 9.5 to about 10.5% by weight lactose anhydrous, and (e) about 0.5 to about 1.5% by weight magnesium stearate.

In another embodiment, the compressed powder comprises: (a) about 78.5 to about 79.5% by weight propafenone; (b) about 4.5 to about 5.5% by weight povidone; (c) about 2.5 to about 3.5% by weight glyceryl behenate; (d) about 11.5 to about 12.5% by weight lactose anhydrous, and (e) about 0.5 to about 1.5% by weight magnesium stearate.

2. Slugs

Slugs are formed by compressing the powder described above. Slugs can take any number of different shapes and sizes, including spherical, cylindrical, cubic and irregular.

In one embodiment, slugs are formed into a shape and size that facilitates the ability to break the slug into pellets with a desired shape and size. In one particular embodiment, at least one surface of the slug is scored to further facilitate the ability to break the slug into pellets with a desired shape and size. Scoring can be effected by any means known in the art. In one embodiment, the scoring is effected in conjunction with the compressing step, such that the compressing step results in a compressed slug with at least once surface that is scored. In one embodiment this is effecting using a tooling set of the present invention, as described in more detail below.

Those skilled in the art will appreciate that the depth of the scoring may be varied based on the composition and dimensions of the slug. In one embodiment, the scoring is up to about 95% of the thickness of the slug. In other embodiments of the invention, the scoring is up to about 20%, up to about 30%, up to about 40%, up to about 50%, up to about 60%, up to about 70%, up to about 75%, up to about 80%, up to about 85%, or up to about 90% of the thickness of the slug. Those skilled in the art also will appreciate that the pattern of the scoring may be varied based on the desired shape and size of the pellets. For example, the scoring may have a waffle pattern, with the individual sections (defining “bits” ) taking any desired shape, including squares, rectangles, trapezoids, diamonds, circles, ovals, and the like.

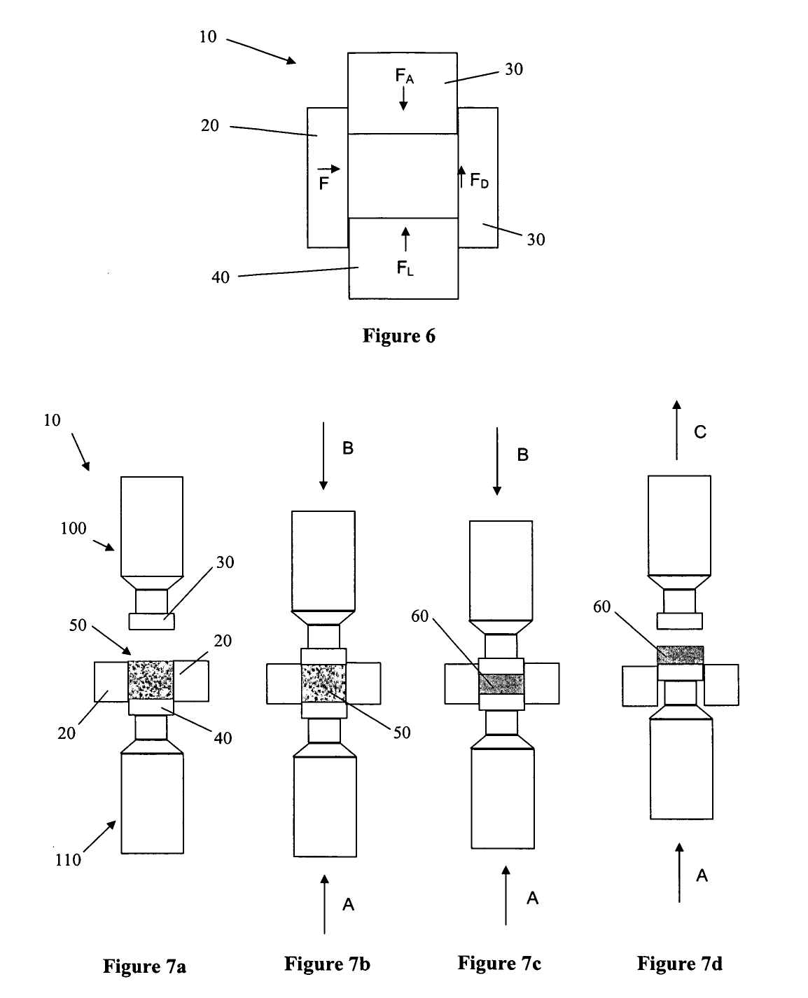

One aspect of the invention provides a tooling set for forming compressed slugs in accordance with the invention. FIG. 6 is a sectional view of a tooling set 10 and the forces that develop in the tooling set during powder compaction. The tooling set 10 includes a die 20, an upper punch 30, and a lower punch 40. As shown in FIG. 6, forces develop during powder compaction, as indicated by axial pressure applied by the upper punch F_{A}, force lost to the die wall F_{D}, radial die wall force F_{R}, and force translated to the lower punch F_{L}.

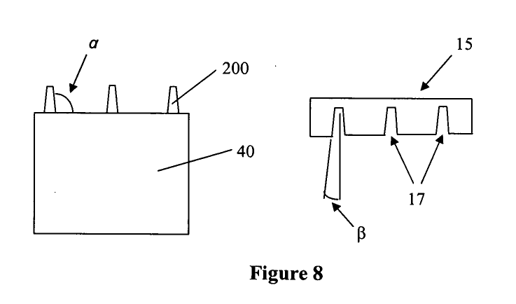

FIG. 7 includes a series of figures to show stages of powder compaction into a slug, according to one embodiment of the invention. FIG. 7a shows a tooling set after powder has been loaded into the tooling set. FIG. 7b shows the tooling set during a precompression stage. FIG. 7c shows the tooling set during the compression stage. FIG. 7d shows the tooling set during ejection of the slug after compression is complete.

FIG. 7a is a sectional view of a tooling set 10 after powder 50 has been loaded into the tooling set 10. The tooling set 10 includes an upper punch set 100 with an upper punch 30, a lower punch set 110 with a lower punch 40, and a die 20. A station for forming compacted powder into slugs may include a single tooling set 10 or a plurality of tooling sets 10. The die 20 and the lower punch 20 can be arranged to form a recess to accommodate powder 50 that is loaded into the tooling set 10.

FIG. 7b is a sectional view of the tooling set 10 during precompression of the powder 50. According to one embodiment, the lower punch set 110 can be moved in the direction indicated by arrow A and the upper punch set 100 can be moved in the direction indicated by arrow B to apply pressure to the powder 50 during precompression. According to another embodiment, the lower punch set 110 can be held stationary while the upper punch set 100 is moved in the direction indicated by arrow B to apply pressure to the powder 50 during precompression. Alternatively, the upper punch set 100 can be held stationary while the lower punch set 110 is moved in the direction indicate by arrow A to apply pressure to the powder 50. When the upper punch set 100 and the lower punch set 110 are moved as indicated, upper punch 30 and lower punch 40 directly apply pressure to the powder 50, which is also constrained by the die 20, causing the powder 50 to be compressed by the tooling set 10.

FIG. 7c is a sectional view of the tooling set 10 during compression of the powder into a slug 60. The movement of the upper and lower punch sets can be as described for any of the embodiments described above.

FIG. 7d is a sectional view of the tooling set 10 during ejection of the slug 60 after compression is complete. According to one embodiment, the upper punch set 100 can be moved in the direction indicated by arrow C to withdraw the upper punch 30 and the lower punch set 110 can be moved in the direction indicated by arrow A to eject the slug 60 from the die 20 so that the slug 60 can be removed from the tooling set 10 and the tooling set 10 can be reset for compression of powder and the production of another slug. Of course, other alternatives for ejecting slugs are contemplated, including moving lower punch set 110 in the direction opposite to that indicated by arrow A to withdraw the lower punch 110 and moving upper punch set 100 in the direction opposite to that indicated by arrow C to eject the slug.

As described above, slugs can be provided with scoring to facilitate breaking of slugs into pellets. Score lines can be produced during compression of powder into slugs, during a subsequent step of compressing powder into slugs, or after the compressed slugs are formed. For example, tooling parts, such as the upper punch and/or lower punch can include a device that produces score lines in slugs during the compression of powder into slugs. The scoring can be adapted for breaking a slug into at least two pellets, for example, by providing a score line that will result in two pellets if the slug is broken along that line. In particular embodiments, the scoring is adapted for breaking a slug into multiple pellets, such as at least about 4, at least about 6, at least about 8, at least about 10, at least about 12, at least about 14, at least about 16, at least about 18, at least about 20 pellets, or more, by providing scoring lines that will result in the desired number of pellets if the slug is broken along the lines. Illustrative tooling parts are described in more detail below.

Slugs may be broken into pellets by various ways such as, for example, impacting the slugs, using a screen to crush the slugs, subjecting the slugs to a low energy milling machine, vibrating the slugs, or by other methods known in the art. In a further example, slugs may be broken apart by hand or by subjecting the slugs to a low energy milling machine operating at about 250-about 500 RPM, including about 300-about 450 RPM, such as about 350-about 400 RPM.

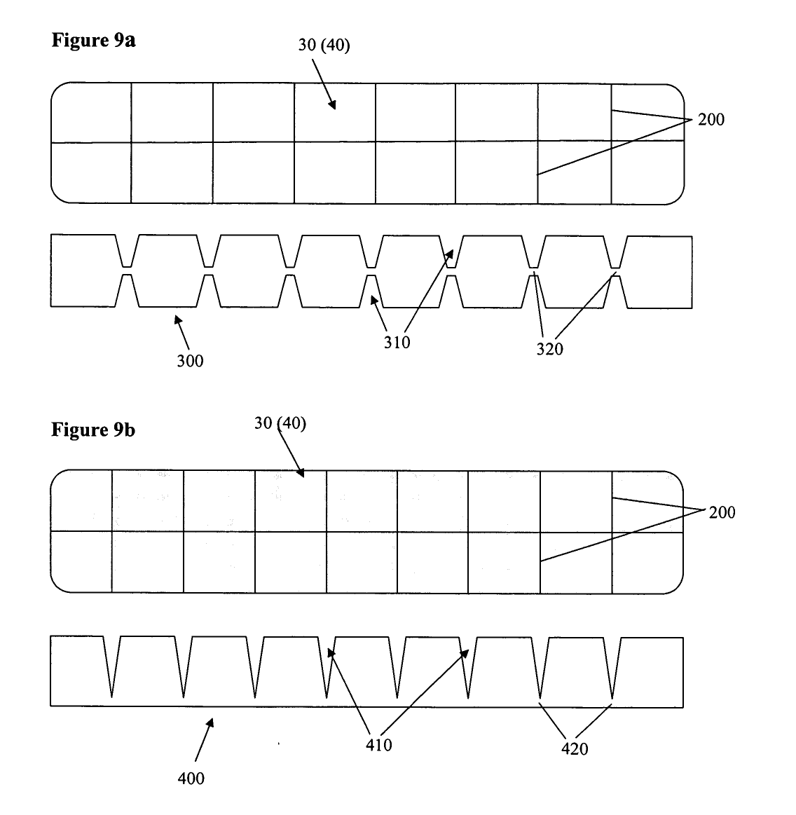

FIG. 8 is a sectional view of a lower punch 40 and an exemplary slug 15 according to one embodiment. The lower punch 40 can include a device 200 for scoring a slug. Such a device 200 for scoring a slug can be a protrusion, ridge, member, pin, or other scoring means known in the art. A device 200 can be discrete or may be continuous across a surface of a tooling part. Although FIG. 8 shows a lower punch 40 with a device 200 for scoring a slug, the upper punch 30 can instead have such a device 200, or both the lower punch 40 and the upper punch 30 can have such a device 200. Tooling parts such as, for example, the lower punch 40 and upper punch 30 can have a single device 200 or a plurality of devices 200 for scoring a slug. The device 200 can include an angle α, as illustrated in FIG. 8, so that a score line 17 formed in a slug 15 by the device 200 will be formed with an angle β that corresponds to the angle α, in which β is an angle between a line tangential to a scored surface of the slug 15 and a line perpendicular to an outer surface of the slug 15. The angle a can be selected for its affect on the process of breaking the slug into pellets, or for its affect on the shape of the pellets, or for other reasons, such as ease or efficiency of compression. In some applications, these considerations may be competing. For example, an angle resulting in a wide scoring pattern may be desired to facilitate breaking, but such an angle may limit the speed of the compression process. For example, the angle β can be selected to be from about 15° to about 90°, including from about 25° to about 80°, from about 35° to about 70°, or from about 45° to about 60°. In one embodiment, the angle β is about 30°. In another embodiment, the angle β is about 60°.

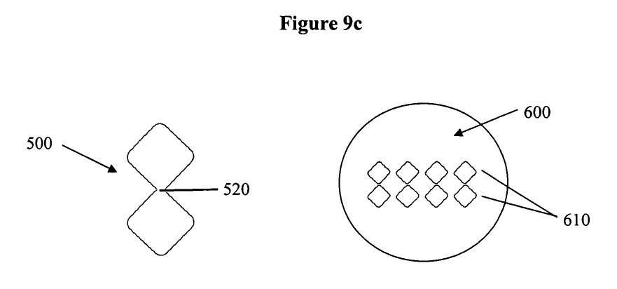

FIGS. 9a-c show a series of tool designs useful for forming slugs in accordance with the invention.

FIG. 9a shows a first tooling design and slug 300 produced with the first tooling design. In the example shown in FIG. 9a, the tooling design is formed on a bottom surface of the upper punch 30 so that the tooling design is formed on the surface of the upper punch 30 that comes in contact with the powder that is compressed within a tooling set. The tooling design can include devices 200 for producing scoring 310 in a slug 300 that is produced by the first tooling design. (The scoring in FIG. 9a is not necessarily drawn to scale.) The lower punch 40 of this tooling set has a tooling design corresponding to the tooling design of the upper punch 30. For example, the upper punch 30 and lower punch 40 can both have devices 200 for producing scoring 310. A slug 300 produced with the first tooling design can be symmetrical in regard to a plane running longitudinally through the center of the slug 300 because the upper punch 30 and lower punch 40 have corresponding tooling designs. The exemplary slug 300 shown in FIG. 9a is designed to break into pellets by breaking apart at members 320 to form pellets of non-uniform size and/or shape.

FIG. 9b shows a second tooling design and slug 400 produced with the second tooling design. In the example shown in FIG. 9b, the tooling design is formed on a bottom surface of the upper punch 30 that comes in contact with the powder that is compressed within a tooling set. The tooling design can include devices 200 for producing scoring 410 in a slug 400 that is produced by the second tooling design. A slug 400 produced with the second tooling design will have scoring 410 formed on one face of the slug 400, as shown in FIG. 9b. (The scoring in FIG. 9b is not necessarily drawn to scale.) In this embodiment, the lower punch 40 has a tooling design that does not include any devices for scoring, or includes devices for scoring to a different depth than that effected by the upper punch. Alternatively, instead of being located on the upper punch, the lower punch 40 of the tooling set can have a tooling design corresponding to the tooling design shown so that the score lines 410 are formed on the bottom of the slug 400 (not shown) instead of the top of the slug, as shown in FIG. 9b. In this latter embodiment, the upper punch has a tooling design that does not include any devices for scoring, or includes devices for scoring to a different depth than that effected by the lower punch. The exemplary slug 400 shown in FIG. 9b is designed to break into pellets by breaking apart at members 420 to form pellets of non-uniform size and/or shape.

FIG. 9c shows a third tooling design and slug 500 produced with the third tooling design. In the example shown in FIG. 9c, the third tooling design includes a plate 600 that includes areas 610 for compressing powder into slug 500. As shown in the example of FIG. 9c, the slug 500 can have the form of two pellets that are joined together by member 520. In this way, slug 500 can be easily broken into two pellets by breaking at member 520. As shown in FIG. 9c, plate 600 will form four slugs 500 for breaking into eight pellets.

Exemplary dimensions of the tools illustrated in FIGS. 9a-c are as follows:

For tooling design 1 (FIG. 9a), the width and height of each individual bit may be about 2.0 mm, and the scoring from the top down and the bottom up may be about 0.8 mm in each direction, such that the total scoring constitutes about 80% of the total height of each bit. The tool as illustrated in FIG. 9a comprises two rows of bits, such that the depth of the tool is about 4.0 mm. The scoring between the rows of bits may be the same as the scoring between each bit, for example, the scoring from the top down and the bottom up may be about 0.8 mm in each direction, such that the total scoring constitutes about 80% of the total height of each bit.

For tooling design 2 (FIG. 9b), the width of each bit may be about 4 mm, and the height of each bit may be about 2 mm. The scoring from the top down may be about 1.4 mm and the scoring from the bottom up may be about 0.2 mm, such that the total scoring constitutes about 80% of the total height of each bit. The tool illustrated in FIG. 9b comprises two rows of bits, such that the depth of the tool is about 2 mm. The scoring between the rows of bits may be the same as the scoring between each bit, for example, the scoring from the top down may be about 1.4 mm and the scoring from the bottom up may be about 0.2 mm, such that the total scoring constitutes about 80% of the total height of each bit.

For tooling design 3 (FIG. 9c), each of the eight boundaries defining areas 610 may be about 2.0 mm.

These designs are illustrative only, and those skilled in the art can design and make other tools suitable for forming slugs in accordance with the present invention.

3. Pellets

In accordance with one aspect of the invention, pellets are formed from the slugs described above by breaking the slugs into pellets. The slugs may be controllably broken into multiple pellets with different surface areas, dissolution rates, and/or release rates, by methods described above.

The pellets can be of any shape. For example, regularly shaped pellets may be cubic, spherical, cylindrical, or any other shape. Irregularly shaped pellets may be for example, primarily cubic, round, spherical, cylindrical, or primarily irregular.

In one particular aspect of the invention, the pellets are non-uniform. That is, the pellets exhibit variation in shape and/or size. According to one embodiment, pellets can have a variation (from the mean) of from about 1% to about 50% (greater than or less than the mean), in diameter, weight or volume, due to the non-uniformity of the size and/or shape of the pellets, including a variation of about 5%, about 10%, about 15%, about 20%, about 25%, about 30%, about 35%, about 40%, about 45%, or about 50% from the mean.

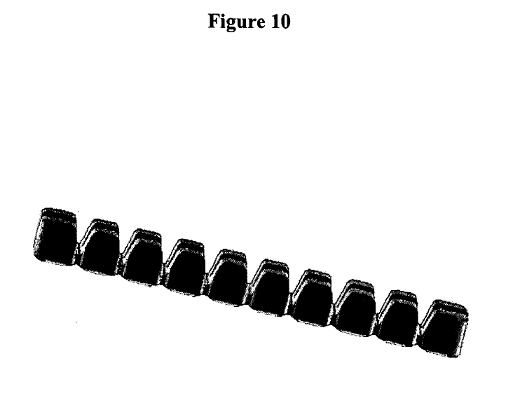

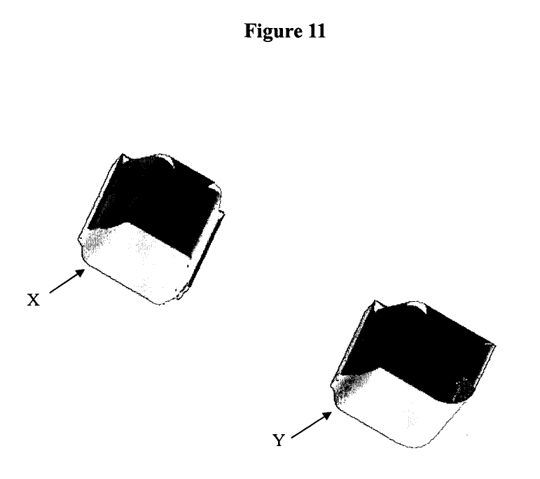

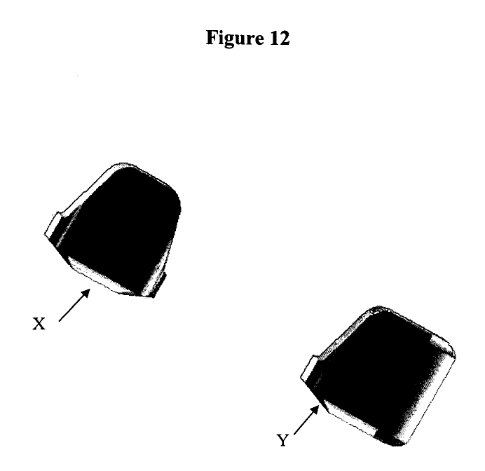

FIGS. 10-12 show exemplary slugs and pellets made with the tooling designs described above. FIG. 10 shows an example of a slug made with the second tooling design of FIG. 9b. FIGS. 11 and 12 show examples of pellets made from a slug produced with the second tooling design of FIG. 9b. These pellets are non-uniform in size and shape. For example, as shown in FIGS. 11 and 12, pellet X and Y are different in size because pellet Y is more square in shape than pellet X, and the pellets are different in shape because pellet X was broken at three places while pellet Y was broken at two places.

In one embodiment, the pellets are of a size suitable for fitting inside a capsule suitable for oral administration. In some embodiments, pellets have dimensions of up to about 10 mm, or up to about 5 mm. In other embodiments, the pellets have dimensions of less than about 5 mm, such as dimensions of from about 1 mm to about 3 mm. For pellets that have a maximum and a minimum diameter, each may independently be up to about 10 mm, up to about 5 mm, less than about 5 mm, or from about 1 mm to about 3 mm.

The pellets may optionally be coated with a pharmaceutically acceptable coating material prior to encapsulation, as discussed in more detail below.

4. Capsules

As noted above, the oral dosage form of the present invention can comprise a capsule comprising the pellets described above. Any pharmaceutically acceptable capsule material may be used for the capsule, such as push-fit capsules made of gelatin or soft, sealed capsules made of gelatin and a plasticizer (such as glycerol or sorbitol), or hard gelatin capsules, or capsules made of hydroxypropylmethyl cellulose (HPMC)

The capsules can comprise the pellets in admixture with one or more other optional ingredients, as discussed herein. Exemplary optional ingredients include, but are not limited to, binding agents, filling agents, lubricating agents, suspending agents, sweeteners, flavoring agents, preservatives, buffers, wetting agents, disintegrants, effervescent agents, stabilizers, or other optional ingredients or excipients, examples of which are given below. Such excipients are known in the art.

The pellets can be encapsulated in the capsule by any means known in the art.

As discussed above, in accordance with one aspect of the invention, the capsules can comprise non-uniform pellets. That is, a given capsule will contain pellets that exhibit variation in shape and/or size, such that not all of the pellets in a given capsule have the same shape and size.

In one embodiment, pellets are selected for inclusion in capsules based on size and/or shape. In one particular embodiment, pellets are selected to have a degree of variability in size and/or shape. Methods for controlling the variation of pellet size and/or shape are known. For example, screening may be used to select pellets within a certain size range. For example, pellets having a size of from about 50% to about 150% of the mean pellet size in the sample can be selected. Alternatively, pellets having a size of from about 80% to about 120% of the mean pellet size in the sample can be selected. In one specific embodiment, pellets having a size of from about 90% to about 110% of the mean pellet size in the sample are selected. In this context, the size can be assessed based on a single dimension, such as diameter, cross-section, weight or volume. Those skilled in the art can select the size and shape of pellets to achieve a desired delivery profile, including a controlled and/or extended delivery profile.

5. Optional Ingredients

In addition to the pharmaceutically active ingredient, the oral dosage forms of the present invention may comprise one or more additional ingredients that are suitable for orally administered compositions. For example, the dosage form may include one or more surfactants, additives, release delaying substances and coatings. These ingredients may be chosen for their effect on the release rate of the active agent, or may be selected for any number of other reasons known to those skilled in the art.

a. Surfactants

Various embodiments of the invention may include a hydrophilic surfactant. Hydrophilic surfactants can be used to provide any of several advantageous characteristics to the compositions, including: increased solubility of the active ingredient in the solid carrier; improved dissolution of the active ingredient; improved solubilization of the active ingredient upon dissolution; enhanced absorption and/or bioavailability of the active ingredient, particularly a hydrophilic active ingredient; and improved stability, both physical and chemical, of the active ingredient. The hydrophilic surfactant can be a single hydrophilic surfactant or a mixture of hydrophilic surfactants, and can be ionic, non-ionic, cationic, anionic, or zwitterionic.

Additionally or alternatively, various embodiments of the invention may include a lipophilic component, such as a lipophilic surfactant, including a mixture of lipophilic surfactants, a triglyceride, or a mixture thereof. The lipophilic surfactant can provide any of the advantageous characteristics listed above for hydrophilic surfactants, as well as further enhancing the function of the surfactants.

Many surfactants suitable for use in oral dosage forms are well known to one of skill in the art, such as polyethoxylkated fatty acids, PEG-fatty acid diesters, PEG-fatty acid mon- and di-ester mixtures, PEG glycerol fatty acid esters, alcohol-oil transesterification products, polyglycerized fatty acids, propylene glycerol fatty acid esters, mixtures of propylene glycol esters-glycerol esters, mono- and diglycerides, sterol and sterol derivatives, polyethylene glycol sorbitan fatty acid esters, polyethylene glycol alkyl ethers, sugar esters, polyethylene glycol alkyl phenols, polyoxyethylene-polyoxypropylene black copolymers, sorbitan fatty acid esters, lower alcohol fatty acid esters, ionic surfactants, unionized ionizable surfactants, and derivatives of fat soluble vitamins.

For compositions of the present invention that include a lipophilic additive, the lipophilic component can be a lipophilic surfactant or a triglyceride. Exemplary triglycerides are those which solidify at ambient room temperature, with or without addition of appropriate additives, or those which in combination with particular surfactants and/or active ingredients solidify at room temperature. Triglycerides suitable for use in the present invention are readily available from commercial sources. Fractionated triglycerides, modified triglycerides, synthetic triglycerides, and mixtures of triglycerides are also within the scope of the invention. Specific examples of suitable triglycerides include glyceryl behenate (also known as Compritol 888 ATO), vegetable oils, fish oils, animal fats, hydrogenated vegetable oils, partially hydrogenated vegetable oils, medium and long-chain triglycerides, and structured triglycerides.

It should be appreciated that several commercial surfactant compositions contain small to moderate amounts of triglycerides, typically as a result of incomplete reaction of a triglyceride starting material in, for example, a transesterification reaction. Such commercial surfactant compositions, while nominally referred to as “surfactants,” may also serve as triglycerides in accordance with the present invention. Examples of commercial surfactant compositions containing triglycerides include some members of the surfactant families Gelucires (Gattefosse), Maisines (Gattefosse), and lmwitors (Huls). Specific examples of these compositions are: Gelucire 44/14 (saturated polyglycolized glycerides); Gelucire 50/13 (saturated polyglycolized glycerides); Gelucire 53/10 (saturated polyglycolized glycerides); Gelucire 33/01 (semi-synthetic triglycerides of C₈-C₈ saturated fatty acids); Gelucire 39/01 (semi-synthetic glycerides); other Gelucires, such as 37/06, 43/01, 35/10, 37/02, 46/07, 48/09, 50/02, 62/05, etc.; Maisine 35-I (linoleic glycerides); and Imwitor 742 (caprylic/capric glycerides).

b. Optional Additives

The oral dosage forms of the present invention may include one or more pharmaceutically acceptable additives, such as those well known in the art. Specific examples include, but are not limited to:

anti-adherents (anti-sticking agents, glidants, flow promoters, lubricants) such as talc, colloidal silicon dioxide, such as Aerosil® 200, magnesium stearate, fumed silica (Carbosil, Aerosil), micronized silica (Syloid No. FP 244, Grace U.S.A.), polyethylene glycols, surfactants, waxes, stearic acid, stearic acid salts, stearic acid derivatives, calcium stearate, silica gel, starch, hydrogenated vegetable oils, sodium benzoate, sodium acetate, leucine, PEG-4000 and magnesium lauryl sulfate;

anticoagulants, such as acetylated monoglycerides;

antifoaming agents, such as long-chain alcohols and silicone derivatives;

antioxidants, such as BHT, BHA, gallic acid, propyl gallate, ascorbic acid, ascorbyl palmitate, 4-hydroxymethyl-2,6-di-tert-butyl phenol, and tocopheryl;

binders (adhesives), i.e., agents that impart cohesive properties to powdered materials through particle-particle bonding, such as various celluloses and cross-linked polyvinylpyrrolidone, matrix binders (dry starch, dry sugars), film binders (PVP, starch paste, celluloses, bentonite, sucrose), and chemical binders (polymeric cellulose derivatives, such as carboxy methyl cellulose, HPC and HPMC; sugar syrups; corn syrup; water soluble polysaccharides such as acacia, tragacanth, guar and alginates; gelatin; gelatin hydrolysate; agar; sucrose; dextrose; and non-cellulosic binders, such as PVP, PEG, vinyl pyrrolidone copolymers, pregelatinized starch, sorbitol, glucose, microcrystalline cellulose, such as Avicel® PH101 and Avicel® PH102, and silicified microcrystalline cellulose (ProSolv SMCC™);

bufferants, where the acid is a pharmaceutically acceptable acid, such as hydrochloric acid, hydrobromic acid, hydriodic acid, sulfuric acid, nitric acid, boric acid, phosphoric acid, acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid and uric acid, and where the base is a pharmaceutically acceptable base, such as an amino acid, an amino acid ester, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, or a salt of a pharmaceutically acceptable cation and acetic acid, acrylic acid, adipic acid, alginic acid, alkanesulfonic acid, an amino acid, ascorbic acid, benzoic acid, boric acid, butyric acid, carbonic acid, citric acid, a fatty acid, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid;

chelating agents, such as EDTA and EDTA salts;

coagulants, such as alginates;

colorants or opaquants, such as titanium dioxide, food dyes, lakes, natural vegetable colorants, iron oxides, silicates, sulfates, magnesium hydroxide and aluminum hydroxide;

coolants, such as halogenated hydrocarbons (e.g., trichloroethane, trichloroethylene, dichloromethane, fluorotrichloromethane), diethylether and liquid nitrogen;

cryoprotectants, such as trehelose, phosphates, citric acid, tartaric acid, gelatin, dextran and mannitol;

pharmaceutically acceptable inert diluents or fillers, such as lactose (such as lactose monohydrate, lactose anhydrous, and Pharmatose® DCL21), mannitol, talc, magnesium stearate, sodium chloride, potassium chloride, citric acid, spray-dried lactose, starch, hydrolyzed starches, directly compressible starch, microcrystalline cellulose (such as Avicel® PH101 and Avicelo PH102), cellulosics, sorbitol, sucrose, glucose, sucrose-based materials, saccharides, calcium sulfate, dibasic calcium phosphate (such as Emcompress®) and dextrose, and/or mixtures of any of the foregoing;

disintegrants or super disintegrants, such as croscarmellose sodium, starch, starch derivatives, corn starch, potato starch, maize starch, modified starches, clays, gums, cellulose, cellulose derivates, alginates, crosslinked polyvinylpyrrolidone, sodium starch glycolate, microcrystalline cellulose, cross-povidone, sodium starch glycolate, and mixtures thereof;

filling agents such as lactose (e.g., lactose monohydrate and lactose anhydrous), and various starches;

hydrogen bonding agents, such as magnesium oxide;

flavorants or desensitizers, such as spray-dried flavors, essential oils and ethyl vanillin;

ion-exchange resins, such as styrene/divinyl benzene copolymers, and quaternary ammonium compounds;

plasticizers, such as polyethylene glycol, citrate esters (e.g., triethyl citrate, acetyl triethyl citrate, acetyltributyl citrate), acetylated monoglycerides, glycerin, triacetin, propylene glycol, phthalate esters (e.g., diethyl phthalate, dibutyl phthalate), castor oil, sorbitol and dibutyl seccate;

preservatives, such as ascorbic acid, boric acid, sorbic acid, benzoic acid, and salts thereof, parabens (e.g., methylparaben, propylparaben), benzyl alcohol, quaternary ammonium compounds such as benzalkonium chloride, potassium sorbate, benzoic acid and its salts, other esters of parahydroxybenzoic acid such as butylparaben, alcohols such as ethyl or benzyl alcohol, phenolic compounds such as phenol;

effervescent agents or effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present;

solvents, such as alcohols, ketones, esters, chlorinated hydrocarbons and water;

sweeteners or flavoring agents, including any natural or artificial sweetener such as maltose, sucrose, glucose, sorbitol, glycerin, dextrins, xylitol, and artificial sweeteners, such as aspartame, acsulfame, saccharine and saccharine salts (e.g., sodium saccharin), cyclamate, Magnasweet® (trademark of MAFCO), bubble gum flavor, and fruit flavors, and the like; and

thickeners (viscosity modifiers, thickening agents), such as sugars, polyvinylpyrrolidone, cellulosics, polymers and alginates.

Additives can also be materials such as proteins (e.g., collagen, gelatin, Zein, gluten, mussel protein, lipoprotein); carbohydrates (e.g., alginates, carrageenan, cellulose derivatives, pectin, starch, chitosan); gums (e.g., xanthan gum, gum arabic); spermaceti; natural or synthetic waxes; carnuaba wax; fatty acids (e.g., stearic acid, hydroxystearic acid); fatty alcohols; sugars; shellacs, such as those based on sugars (e.g., lactose, sucrose, dextrose) or starches; polysaccharide-based shellacs (e.g., maltodextrin and maltodextrin derivatives, dextrates, cyclodextrin and cyclodextrin derivatives); cellulosic-based shellacs (e.g., microcrystalline cellulose, sodium carboxymethyl cellulose, hydroxypropylmethyl cellulose, ethyl cellulose, hydroxypropyl cellulose, cellulose acetate, cellulose nitrate, cellulose acetate butyrate, cellulose acetate trimellitate, carboxymethylethyl cellulose, hydroxypropylmethyl cellulose phthalate); inorganics, such as dicalcium phosphate, hydroxyapitite, tricalcium phosphate, talc and titania; polyols, such as mannitol, xylitol and sorbitol; polyethylene glycol esters; and polymers, such as alginates, poly(lactide coglycolide), gelatin, crosslinked gelatin, and agar-agar.

It should be appreciated that there is considerable overlap between the above-listed additives in common usage, since a given additive is often classified differently by different practitioners in the field, or is commonly used for any of several different functions. Thus, the above-listed additives should be taken as merely exemplary, and not limiting, of the types of additives that can be included in compositions of the present invention. The amounts of such additives can be readily selected and optimized by one skilled in the art, according to the particular properties desired.

c. Release Delaying Substance

The oral dosage form also may include a release delaying substance to further control and/or extend the delivery of the active ingredient. In one embodiment such a substance is included in the compressed powder. In accordance with that embodiment, the delayed release substance may comprise from about 1% up to about 50% of the weight of the compressed powder. In another embodiment, such a substance is included in a coating provided on the pellets. In one specific embodiment, povidone is used as a delayed release substance.

Extended release and targeted delayed release coatings for oral dosage forms are known in the art, and include those described in U.S. Pat. Nos. 5,622,721 and 5,686,105, the disclosures of which are incorporated herein by reference in their entirety. Substances such as sodium carboxymethyl cellulose, hydroxypropylmethyl cellulose, ethyl cellulose, hydroxypropyl cellulose, cellulose acetate, polyethylene oxide, sodium alginate, glyceryl behanate, and acrylic polymers may be used.

d. Coatings

As noted above, the pellets can be provided with a coating prior to encapsulation. Additionally or alternatively, the slugs can be provided with a coating prior to formation of the pellets. As a further option, the capsules can be provided with a coating. Examples of suitable coatings include but are not limited to seal coatings, enteric coatings, extended release coatings, and targeted delayed release coatings. These coatings are known in the art, and described briefly below.

Seal coating, or coating with isolation layers: Thin layers can be applied for variety of reasons, including for particle porosity reduction, to reduce dust, for chemical protection, to mask taste, to reduce odor, to minimize gastrointestinal irritation, etc. The isolating effect is proportional to the thickness of the coating. Water soluble cellulose ethers are exemplary for this application. HPMC and ethyl cellulose in combination, or Eudragit E100, may be particularly suitable for taste masking applications. Traditional enteric coating materials listed elsewhere can also be applied to form an isolating layer.

Extended release coating: The term “extended release coating” as used herein means a coating designed to provide delivery over an extended period of time. In one embodiment, the extended release coating is a pH-independent coating formed of, for example, ethyl cellulose, hydroxypropyl cellulose, methylcellulose, hydroxymethyl cellulose, hydroxyethyl cellulose, acrylic esters, or sodium carboxymethyl cellulose. Various extended release dosage forms can be readily designed by one skilled in art to achieve delivery to both the small and large intestines, to only the small intestine, or to only the large intestine, depending upon the choice of coating materials and/or coating thickness. Other extended release and targeted delayed release coatings useful in the present invention are described more completely in U.S. Pat. Nos. 5,622,721 and 5,686,105, the disclosures of which are incorporated herein by reference in their entirety.

Enteric coating: The term “enteric coating” as used herein relates to a mixture of pharmaceutically acceptable excipients which is applied to the pellets to protect the pellets during the digestive process. The enteric coating may be applied through an aqueous dispersion or after dissolving in appropriate solvent. Specific enteric coatings may be selected based on any combination of the following criteria:

1. resistance to dissolution and disintegration in the stomach;

2. impermeability to gastric fluids and drug/carrier/enzyme while in the stomach;

3. ability to dissolve or disintegrate rapidly at the target intestine site;

4. physical and chemical stability during storage;

5. non-toxicity;

6. easy application as a coating (substrate friendly); and

7. economical practicality.

Examples of suitable eneteric coatings include but are not limited to anionic polymers exhibiting a pH-dependent solubility profile, such as anionic carboxylic polymers. Further examples, include, but are not limited to those listed below:

(1) Shellac, also called purified lac, a refined product obtained from the resinous secretion of an insect. This coating dissolves in media of pH>7.

(2) Acrylic polymers: The performance of acrylic polymers (primarily their solubility in biological fluids) can vary based on the degree and type of substitution. Examples of suitable acrylic polymers include methacrylic acid copolymers and ammonio methacrylate copolymers. The Eudragit series E, L, S, RL, RS and NE (Rohm Pharma) are available as solubilized in organic solvent, aqueous dispersion, or dry powders. The Eudragit series RL, NE, and RS are insoluble in the gastrointestinal tract but are permeable and are used primarily for extended release. The Eudragit series E dissolve in the stomach. The Eudragit series L, L-30D and S are insoluble in stomach and dissolve in the intestine.

(3) Cellulose Derivatives: Examples of suitable cellulose derivatives include but are not limited to ethyl cellulose and reaction mixtures of partial acetate esters of cellulose with phthalic anhydride. The performance can vary based on the degree and type of substitution. Cellulose acetate phthalate (CAP) dissolves in pH>6. Aquateric (FMC) is an aqueous based system and is a spray dried CAP psuedolatex with particles. Other components in Aquateric can include pluronics, Tweens, and acetylated monoglycerides; cellulose acetate trimellitate (Eastman); methylcellulose (Pharmacoat, Methocel); and hydroxypropylmethyl cellulose phthalate (HPMCP). The performance can vary based on the degree and type of substitution. HP-50, HP-55, HP-55S, HP-55F grades are suitable, as is hydroxypropylmethyl cellulose succinate (HPMCS; AQOAT (Shin Etsu)). The performance can vary based on the degree and type of substitution. Suitable grades include AS-LG (LF), which dissolves at pH 5, AS-MG (MF), which dissolves at pH 5.5, and AS-HG (HF), which dissolves at higher pH. These polymers are offered as granules, or as fine powders for aqueous dispersions; and

(4) Poly Vinyl Acetate Phthalate (PVAP). PVAP dissolves in pH>5, and it is much less permeable to water vapor and gastric fluids.

Other exemplary coating materials are made from opadry colors or with coating material that comprises a pH dependent polymer and/or a pH independent polymer. Specific examples of pH-dependent polymers include: methacrylic acid/methyl methacrylate copolymers (Eudragit L 100, Eudragit S 100), methacrylic acid/ethyl acrylate copolymers (Eudragit L 30D 55), methacrylic acid/methyl acrylate and methyl methacrylate copolymers (Eudragit FS 30D), cellulose acetate phthalate, cellulose acetate trimellitate, hydroxypropyl methylcellulose acetate succinate, hydroxypropyl methylcellulose phthalate, povinyl acetate phthalate and shellac. Preferred pH-independent polymer are ethylcellulose, methacrylic ester copolymers (Eudragit NE 30D), and ammonio methacrylate copolymers (Eudragit RL 30D and Eudragit RS 30D).

Combinations of the above materials can also be used.

The coating can, and often does, comprise a plasticizer and possibly other coating excipients such as colorants, talc, and/or magnesium stearate, which are well known in the art. Suitable plasticizers include but are not limited to triethyl citrate (Citroflex 2), triacetin (glyceryl triacetate), acetyl triethyl citrate (Citroflec A2), Carbowax 400 (polyethylene glycol 400), diethyl phthalate, tributyl citrate, acetylated monoglycerides, glycerol, fatty acid esters, propylene glycol, and dibutyl phthalate. In particular, anionic carboxylic acrylic polymers usually will comprise about 10 to about 25% by weight of a plasticizer, especially dibutyl phthalate, polyethylene glycol, triethyl citrate and triacetin.

Colorants, detackifiers, surfactants, antifoaming agents, lubricants, stabilizers such as hydroxypropylcellulose, acid/base also may be included in the coatings. Such ingredients may solubilize or disperse the coating material, and improve coating performance and the coated product.

A particularly suitable methacrylic copolymer is Eudragit L®, particularly L-30D® and Eudragit 100-55®, manufactured by Rohm Pharma (Germany). In Eudragit L-30D®, the ratio of free carboxyl groups to ester groups is approximately 1:1. Further, the copolymer is known to be insoluble in gastrointestinal fluids having pH below 5.5, generally 1.5-5.5, i.e., the pH generally present in the fluid of the upper gastrointestinal tract, but readily soluble or partially soluble at pH above 5.5, i.e., the pH generally present in the fluid of lower gastrointestinal tract.

Another methacrylic acid polymer which is suitable for use as a coating is Eudragit S®, manufactured by Rohm Pharma (Germany). Eudragit® S differs from Eudragit® L-30-D only insofar as the ratio of free carboxyl groups to ester groups is approximately 1:2. Eudragit S is insoluble at pH below 5.5, but unlike Eudragit L-30-D, is poorly soluble in gastrointestinal fluids having pH of 5.5-7.0, such as is present in the small intestine media. This copolymer is soluble at pH 7.0 and above, i.e., the pH generally found in the colon. Eudragit® S can be used alone as a coating to provide delivery of beginning at the large intestine via a delayed release mechanism. In addition, Eudragit® S, being poorly soluble in intestinal fluids below pH 7, can be used in combination with Eudragit® L-30-D, soluble in intestinal fluids above pH 5.5, to effect a delayed release composition. The more Eudragit® L-30D used the more proximal release and delivery begins, and the more Eudragit® S used, the more distal release and delivery begins. Both Eudragit® L-30-D and Eudragit® S can be substituted with other pharmaceutically acceptable polymers with similar pH solubility characteristics.

Thus, exemplary coating materials include shellac, acrylic polymers, cellulosic derivatives, polyvinyl acetate phthalate, and mixtures thereof. More specific exemplary coating materials include Eudragit® series E, L, S, RL, RS, NE, L , L300, S, 100-55, cellulose acetate phthalate, Aquateric, cellulose acetate trimellitate, ethyl cellulose, hydroxypropyl methyl cellulose phthalate, hydroxypropyl methyl cellulose succinate, poly vinyl acetate phthalate, and Cotteric. Further examples include Eudragit® series L, L300, S, L100-55, cellulose acetate phthalate, Aquateric, ethyl cellulose, hydroxypropyl methyl cellulose phthalate, hydroxypropyl methyl cellulose succinate, poly vinyl acetate phthalate, and Cotteric.

Conventional coating techniques such as spray or pan coating can be employed to apply coatings in accordance with the present invention. The coating thickness can be selected to optimize the release rate, and to protect the oral dosage form (or its constituent pellets) until the desired site of delivery in the intestinal tract is reached.

The following examples are intended to further describe the invention by way of illustration only, and should not be construed as limiting the scope of the invention in any way. Throughout the specification, any and all references to a publicly available document, including a U.S. Patent, are specifically incorporated by reference.

### EXAMPLE 1

### Reference

### Doxycycline Hyclate Delayed Release Capsules

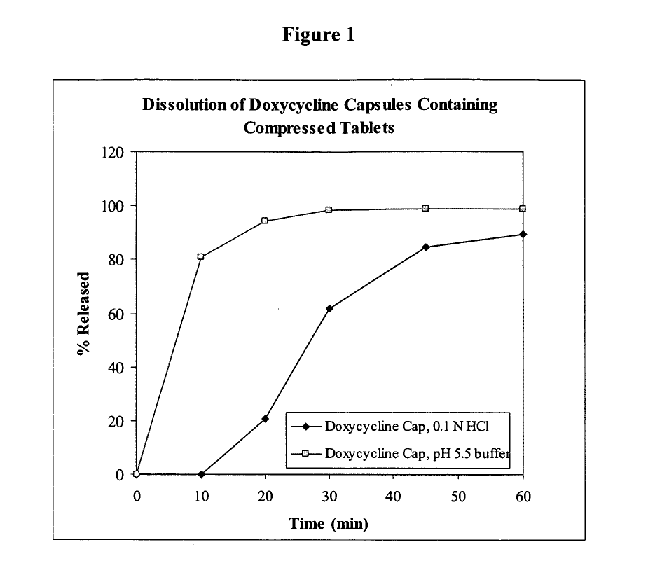

Capsules comprising delayed release tablets comprising doxycycline hyclate were produced. The composition of the tablet is listed in Table 1. The manufacturing process included blending and milling of the tablet ingredients. Additional blending and lubrication was performed to obtain the final blend. The final blend was compressed into mini-tablets (4.5 mm diameter, round). Each tablet comprised 25 mg of the active ingredient doxycycline. A coating was applied to the tablets and the tablets were encapsulated into empty capsules. Three or four tablets were filled into capsules to produce doxycycline hyclate capsules with a final dosage of 75 mg or 100 mg. The typical dissolution profile is shown in FIG. 1.

The dissolution profiles were obtained by standard methods. The results illustrated in FIG. 1 reflect dissolution tests conducted under two different conditions: (1) USP Apparatus I (basket), 50 rpm, 900 mL of 0.06 N HCl and (2) USP Apparatus I (basket), 50 rpm, 1000 mL of pH 5.5 neutralized phthalate buffer. Both tests were performed at 37° C. The capsules were added to baskets of the apparatus, subject to rotation at the listed rate, and the dissolution was measured over time.

TABLE 1
Composition of Doxycycline Hyclate (mini) Tablets for encapsulation

| | |
|---|---|
| mg/tablet | Ingredients |

Part I Compressed Tablets

| | |
|---|---|
| 28.855 | Doxycycline Hyclate, USP |
| =25.00 | Doxycycline Anhydrous |
| 11.830 | Microcrystalline Cellulose, NF |
| 10.000 | Microcrystalline Cellulose, NF |
| 9.600 | Sodium Starch Glycolate, NF |
| 0.500 | Colloidal Silicon Dioxide, NF |
| 2.400 | Talc, USP |
| 0.600 | Magnesium Stearate, NF |

Part II Coating

| | |
|---|---|
| 1.400 | Hypromellose Phthalate, NF |
| 0.426 | Talc, USP |

### EXAMPLE 2

### Reference

### Omeprazole and Esomeprazole Delayed Release Capsules

For development of delayed release esomeprazole capsules, mini-tablets with 4.76 mm diameter were compressed and then coated with enteric polymer in alkaline solution. Each tablet comprised 10 mg of the active pharmaceutical ingredient. Two of four coated tablets were filled into capsules to produce capsules at 20 mg or 40 mg strengths. Table 2 shows the composition of esomeprazole (mini) tablets.

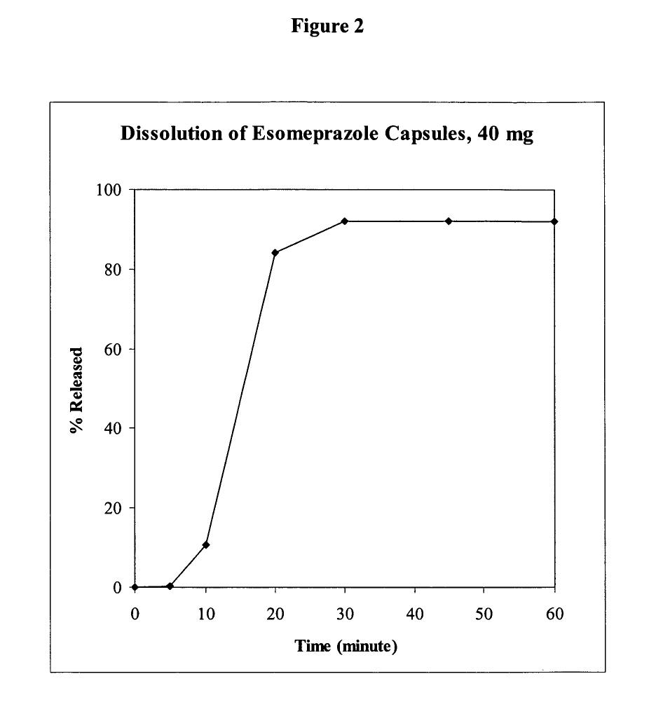

For an acid resistant study, tablets were soaked in 0.1 N HCl for 2 hours. Only negligent amounts of omeprazole or esomeprazole were degraded. On the other hand, the dissolution of the esomeprazole capsules in pH 6.8 was fast. The dissolution profile using USP Apparatus II, 100 rpm, in pH 6.8 buffer is shown in FIG. 2.

TABLE 2
Composition of Esomeprazole (mini) Tablets for encapsulation

| | |
|---|---|
| Ingredient | Mg/tablet |

Part I Compressed Tablets

| | |
|---|---|
| Esomeprazole | 10.00 |
| Magnesium Carbonate | 1.25 |
| Lactose DCL 15 | 26.00 |
| Microcrystalline Cellulose, NF | 15.25 |
| Talc, USP | 1.50 |
| Mg-Stearate, NF | 1.00 |

Tablet Coating

| | |
|---|---|
| Hypromellose Phthalate, NF | 3.700 |
| Talc | 1.864 |

### EXAMPLE 3

### Propafenone HCl Capsules Comprising Granules

Propafenone HCl capsules comprising granules were prepared from a blend of the ingredients listed in Table 3. Propofenone HCl was granulated to form a powder, which was sprayed with an aqeous solution of povidone. That mixture was sprayed with an aqueous suspension of ethyl cellulose, resulting in wet granules. After drying, the granules were milled and then blended with magnesium stearate (as a lubricant) to form a final blend. The final blend was compressed using a roller compactor, resulting in a compressed powder in the form of a sheet or “slab.” The compressed sheet was milled into granules. The resulting granules were screened to obtain granules of the desired size, and granules of the desired size were placed into capsules.

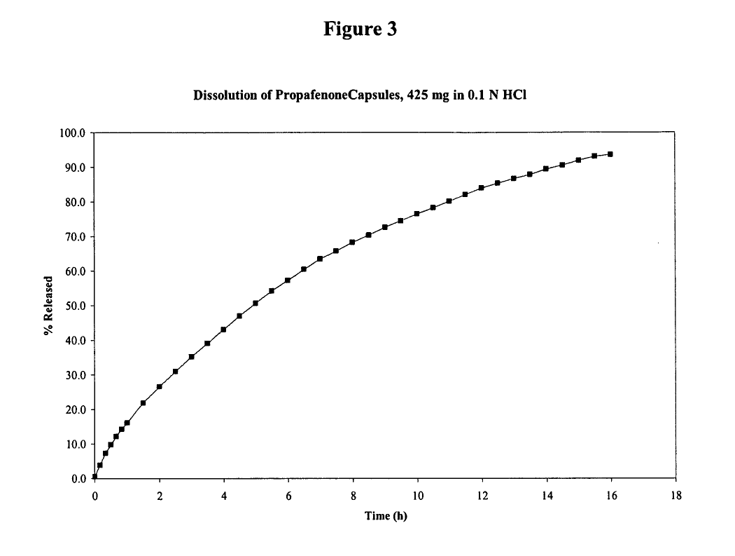

The dissolution profile of the capsules in 0.1 N HCl was tested using USP apparatus II (paddle) at 50 rpm. The dissolution profile of the capsules is shown in FIG. 3. As shown in that figure, propafenone HCl was gradually released from the capsule, with an extended release profile that lasted several hours.

This process produces granules with a wide size variation and, as a result, achieves an actual yield of only about 10-15% (based on the weight of the final blend) because of the production of granules (and waste “powder”) that fall outside of the desired size.

TABLE 3
Composition of Propafenone HCl Granules

| Ingredient | Amount % |
|---|---|
| Propafenone HCl | 95.50 |
| Povidone | 3.00 |
| Ethyl Cellulose | 1.00 |
| Magnesium Stearate | 0.50 |

### EXAMPLE 4

### Propafenone HCl Capsules Comprising Non-Uniform Pellets

Propafenone HCl capsules comprising non-uniform pellets were prepared from a blend of the ingredients listed in Table 3 above. The manufacturing process used to obtain the final blend was the same as described above in Example 3. The final blend was compressed into slugs using a tablet press equipped with a tooling design as described above. See FIG. 9B for tooling. The slugs were milled with a low energy mill into granules. Granules of a desired size were selected by screening. In particular, granules with a diameter greater than 2.3 mm or less than 1.7 mm were removed. Granules of the desired size were placed into capsules.

The dissolution profile ofthe capsules in 0.1 N HCl was tested using USP apparatus II (paddle) at 50 rpm. The dissolution profile was largely identical to that shown in FIG. 3. That is, like the capsules of Example 3, the capsules of example 4 gradually released propafenone HCI, with an extended release profile that lasted several hours.

In contrast to the low effective yield of Example 3 above, this general process has resulted in effective yields of up to about 90% yield, based on the weight of the final blend.

### EXAMPLE 5

To assess the characteristics of different formulations of propafenone HCl capsules under different conditions, powder blends with the compositions of Formula A and Formula B of Table 4 were prepared. Propafenone HCL was mixed with either ethyl cellulose or glyceryl behemate before being sprayed with an aqueous solution of povidone. The formulations were then dried and milled before the addition of lactose anhydrous, followed by magnesium stearate, to obtain the final blends. The final blends were compressed into slugs and broken into pellets as described in Example 4 above.

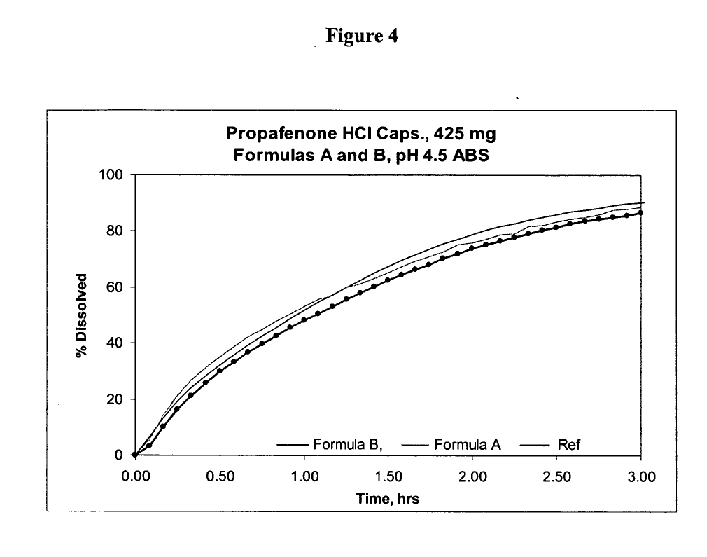

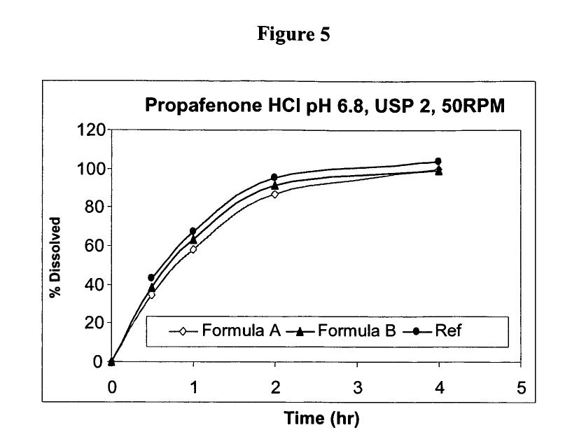

The dissolution profiles for Formulas A and B were assessed using USP Apparatus II (paddle) at 50 rpm at pH 4.5 (FIG. 4) and 6.8 (FIG. 5). Rythmol SR® was used as a reference compound.

TABLE 4
Formulation (For 425 mg Strength)

| | Formula A | | Formula B | |
|---|---|---|---|---|
| Ingredient | Mg/cap | % | Mg/cap | % |
| Propafenone HCl | 425.00 | 79.00 | 425.00 | 79.00 |
| Povidone | 26.90 | 5.00 | 26.90 | 5.00 |
| Ethyl Cellulose | 26.90 | 5.00 | — | — |
| Glyceryl Behenate, NF | — | — | 16.14 | 3.00 |
| (Compritol 888 ATO) | | | | |
| Lactose Anhydrous, NF | 53.80 | 10.00 | 64.56 | 12.00 |
| (Pharmatose DCL-21) | | | | |
| Magnesium Stearate | 5.38 | 1.00 | 5.38 | 1.00 |
| Total | 537.98 | 100.00 | 537.98 | 100.00 |

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

What is claimed is:

1. An oral dosage form of a pharmaceutically active ingredient comprising:
(a) an outer capsule; and
(b) non-uniform pellets, having a non-uniform shape and/or size, comprised within the capsule,
wherein the pellets comprise a compressed powder comprising a pharmaceutically active ingredient.

2. The oral dosage form of claim 1, wherein the active ingredient is selected from the group consisting of doxycycline, omeprazole, esomeprazole, and propafenone.

3. The oral dosage form of claim 1, wherein the non-uniform pellets have a diameter of from about 1 mm to about 3 mm.

4. The oral dosage form of claim 1, wherein the non-uniform pellets have a maximum and a minimum diameter, each of which is independently from about 1 mm to about 3 mm.

5. The oral dosage form of claim 1, wherein the non-uniform pellets are primarily spherical, cubic, cylindrical, irregular, or a combination thereof.

6. The oral dosage form of claim 1, wherein the non-uniform pellets are coated with a pharmaceutically acceptable coating material.

7. The oral dosage form of claim 6, wherein the coating material comprises Opadry colors.

8. The oral dosage form of claim 7, wherein the coating material comprises a material selected from a pH-dependent polymer and a pH-independent polymer.

9. The oral dosage form of claim 8, wherein the pH-dependent polymer is selected from the group consisting of methacrylic acid/methyl methacrylate copolymers (Eudragit L 100, Eudragit S 100), methacrylic acid/ethyl acrylate copolymers (Eudragit L 30D 55), methacrylic acid/methyl acrylate and methyl methacrylate copolymers (Eudragit FS 30D), cellulose acetate phthalate, cellulose acetate trimellitate, hydroxypropyl methylcellulose acetate succinate, hydroxypropyl methylcellulose phthalate, povinyl acetate phthalate and shellac.

10. The oral dosage form of claim 8, wherein the pH-independent polymer is selected from the group consisting of ethylcellulose, methacrylic ester copolymers, and ammonio methacrylate copolymers.

11. The oral dosage form of claim 2, wherein the pellets are made by a process comprising: (a) compressing a powder comprising the pharmaceutically active ingredient into compressed slugs; and (b) breaking the slugs into non-uniform pellets, having a non-uniform shape and/or size.

12. The oral dosage form of claim 11, wherein the compressing step comprises roller compaction.

13. The oral dosage from of claim 11, wherein the process further comprises, subsequent to step (a) and prior to step (b), scoring at least one surface of the slugs.

14. The oral dosage from of claim 11, wherein the compressing step results in the formation of compressed slugs comprising scoring on at least one surface.

15. The oral dosage form of claim 14, wherein the depth of the scoring comprises up to about 95% of the thickness of the slugs.

16. The oral dosage form of claim 2, wherein the powder comprises:
(a) about 78.5 to about 79.5% by weight propafenone;
(b) about 4.5 to about 5.5% by weight povidone;
(c) about 4.5 to about 5.5% by weight ethyl cellulose;
(d) about 9.5 to about 10.5% by weight lactose anhydrous, and
(e) about 0.5 to about 1.5% by weight magnesium stearate.

17. The oral dosage form of claim 2, wherein the powder comprises:
(a) about 78.5 to about 79.5% by weight propafenone;
(b) about 4.5 to about 5.5% by weight povidone;
(c) about 2.5 to about 3.5% by weight glyceryl behenate;
(d) about 11.5 to about 12.5% by weight lactose anhydrous; and
(e) about 0.5 to about 1.5% by weight magnesium stearate.

18. An oral dosage form of a pharmaceutically active ingredient comprising:
(a) about 78.5 to about 79.5% by weight propafenone;
(b) about 4.5 to about 5.5% by weight povidone;
(c) about 4.5 to about 5.5% by weight ethyl cellulose;
(d) about 9.5 to about 10.5% by weight lactose anhydrous, and
(e) about 0.5 to about 1.5% by weight magnesium stearate.

19. An oral dosage form of a pharmaceutically active ingredient comprising:
(a) about 78.5 to about 79.5% by weight propafenone;
(b) about 4.5 to about 5.5% by weight povidone;
(c) about 2.5 to about 3.5% by weight glyceryl behenate;
(d) about 11.5 to about 12.5% by weight lactose anhydrous; and
(e) about 0.5 to about 1.5% by weight magnesium stearate.

20. A method of producing an oral dosage form of a pharmaceutically active ingredient comprising:
(a) compressing a powder comprising a pharmaceutically active ingredient into slugs,
(b) breaking the slugs into non-uniform pellets, having a non-uniform shape and/or size, and
(c) encapsulating the non-uniform pellets in a capsule.

21. The method of claim 20, wherein the compressing step comprises roller compaction.

22. The method of claim 20, further comprising, subsequent to step (a) and prior to step (b), scoring at least one surface of the slugs.

23. The method of claim 20, wherein the compressing step results in the formation of compressed slugs comprising scoring on at least one surface.

24. The method of claim 23, wherein the depth of the scoring comprises up to about 95% of the thickness of the slugs.

25. The method of claim 20, wherein the non-uniform pellets have a diameter of from about 1 mm to about 3 mm.

26. The method of claim 20, wherein the non-uniform pellets have a maximum and a minimum diameter, each of which is independently from about 1 mm to about 3 mm.

27. The method of claim 20, wherein the non-uniform pellets are primarily spherical, cubic, cylindrical, irregular, or a combination thereof.

28. The method of claim 20, further comprising, subsequent to step (b) and prior to step (c), coating the non-uniform pellets with a pharmaceutically acceptable coating material.

29. The method of claim 20, wherein the active ingredient is selected from the group consisting of doxycycline, omeprazole, esomeprazole, and propafenone.

30. The method of claim 20, wherein the powder comprises:
(a) about 78.5 to about 79.5% by weight propafenone;
(b) about 4.5 to about 5.5% by weight povidone;
(c) about 4.5 to about 5.5% by weight ethyl cellulose;
(d) about 9.5 to about 10.5% by weight lactose anhydrous, and
(e) about 0.5 to about 1.5% by weight magnesium stearate.

31. The method of claim 20, wherein the powder comprises:
(a) about 78.5 to about 79.5% by weight propafenone;
(b) about 4.5 to about 5.5% by weight povidone;
(c) about 2.5 to about 3.5% by weight glyceryl behenate;
(d) about 11.5 to about 12.5% by weight lactose anhydrous; and
(e) about 0.5 to about 1.5% by weight magnesium stearate.

32. The method of claim 20, further comprising, subsequent to step (b) and prior to step (c), screening the pellets for a desired shape and/or size profile.

33. An apparatus for making compressed powder slugs that are scored for breaking into pellets, comprising:
tooling that is adapted to compress powder into a slug, wherein at least one surface of the tooling is adapted to form scoring on at least one surface of the slug, for breaking the slug into pellets.

34. The apparatus of claim 33, wherein the tooling comprises a die, a lower punch, and an upper punch.

35. The apparatus of claim 34, wherein at least one of the upper punch and the lower punch is adapted to form scoring on a surface of the slug.

36. The apparatus of claim 35, wherein both the upper punch and the lower punch are adapted to form scoring on upper and lower surfaces of the slug.

37. The apparatus of claim 36, wherein the upper punch and the lower punch are adapted to form scoring of different depths on upper and lower surfaces of the slug, respectively.

38. The apparatus of claim 33, wherein the scoring is adapted for breaking the slug into at least two pellets.

39. The apparatus of claim 33, wherein the scoring is adapted for breaking the slug into at least twenty pellets.

40. The apparatus of claim 33, wherein the tooling is adapted to form a single slug.

41. The apparatus of claim 33, wherein the tooling is adapted to form multiple slugs.

42. A punch for making compressed powder slugs that are scored for breaking into pellets, comprising:
a device adapted to form scoring on a surface of the slug, for breaking the slug into pellets.

43. The punch of claim 42, wherein the device is adapted to form scoring with an angle of from about 15° to about 90° between a line that is tangential to the surface of the slug that comprises the scoring and a line perpendicular to an outer surface of the slug.
